(19) United States
(12) Patent Application Publication
(10) Pub. No.: US 2009/0186007 A1
(43) Pub. Date: Jul. 23, 2009

(54) USE OF CELLS DERIVED FROM ADIPOSE TISSUE FOR THE PREPARATION OF AN ANTI-TUMOR MEDICAMENT

(76) Inventors: Pierre Cordelier, Toulouse (FR); Louis Buscail, Toulouse (FR); Louis Casteilla, Escalquens (FR); Beatrice Cousin-Delarue, Nailloux (FR); Luc Penicaud, Toulouse (FR); Jean-Marie Peron, Toulouse (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/301,343

(22) PCT Filed: May 18, 2007
(86) PCT No.: PCT/FR2007/000847

§ 371 Date: Jan. 6, 2009

(30) Foreign Application Priority Data
May 18, 2006 (FR) 0604443

(51) Int. Cl. A61K 35/12; A61P 35/00; G01N 33/50

(52) U.S. Cl. 424/93.21; 424/93.7; 435/4

(57) ABSTRACT

Use of cells isolated from extramedullary white adipose tissue, selected from the group consisting of the stromal-vascular fraction and a subpopulation of said stromal-vascular fraction consisting of adherent cells, for the preparation of an anti-tumor medicament.

The present invention relates to the use of cells derived from adipose tissue for the preparation of a medicament with antitumor action.

The effective treatment of cancers remains one of the major challenges in medicine today.

The effectiveness of conventional surgical treatments or cytolytic treatments (chemotherapy or radiotherapy) remains very limited in many cancers. In fact, certain cancers which occur with high frequency (gastrointestinal tract, in particular) are always difficult to treat given their mass aspect which is not favorable to the diffusion of therapeutic agents. The treatment of said cancers therefore in the first place requires surgical excision, followed by chemotherapy optionally combined with radiotherapy. However, in cases where surgery is not or is no longer possible, an alternative chemotherapy which is effective without being toxic is necessary. However, the available products have limited effectiveness or unacceptable toxicity. There exists therefore a need for new anticancer treatments, in order to have a sufficiently broad range of treatments, to thus increase the chances of recovery.

In the case of cancers of the gastrointestinal tract (esophagus, stomach, small intestine, large intestine, colon) and of its ancillary glands (liver, gall bladder, pancreas), for example, the lack of curative treatment is proven, when surgical excision is not or is no longer possible. In fact, the existing treatments, which are essentially based on chemotherapy (5-fluorouracil or 5-FU, gemcitabine, etc.) and radiotherapy, have a weak impact on survival and are especially used for palliative purposes, in particular in the case of colorectal, gastric and pancreatic cancers (for a review, Diaz-Rubio, The Oncologist, 2004, 9, 282-294).

In addition to the lack of effective treatments, the toxicity of the chemotherapeutic agents normally used (fluorouracil, folinic acid, platinum derivatives, such as oxaliplatin, mitomycin C, for example) and the side effects associated with these treatments represent another major drawback.

New anticancer agents have been tested, in particular in gastrointestinal tract cancers, such as pemetrexed (antifolate: cytotoxic agent), vaccines, kinase inhibitors (byrostatin, UCN-01, flavopiridol, CI-1040), EGFR inhibitors (cetuximab, gefitinib, GW572016, CI-1033, erlotinib) and VEGF inhibitors (bevacizumab, PTK787/ZK 222584, angiozyme, ZD6474).

For example:
cetuximab is a chimeric monoclonal antibody which binds specifically to the extracellular domain of the human epidermal growth factor receptor (EGFR) and inhibits the proliferation of tumor cells expressing EGFR and induces apoptosis.
bevacizumab is a monoclonal IgG1 antibody which binds to VEGF (Vascular Endothelial Growth Factor) and as a result inhibits VEGF binding to its receptors, Flt-1 (VEGFR-1) and KDR (VEGFR-2), located at the surface of endothelial cells both in vitro and in vivo. It comprises a constant portion of human origin and a variable portion of murine origin.

The results obtained with these new products alone or in combination do not show any improvement compared with the prior treatments, in particular in the treatment of digestive tract cancers, both from the efficacy and toxicity point of view.

In the case of pancreatic cancer, which represents the fifth most common cause of death from cancer in western countries, the physician has even fewer options; in addition to pancreatic cancer being aggressive and often showing clinical signs late, the prognosis thereof is very poor (5-year survival less than 3.5%); in addition, surgical excision is possible only in 10 to 15% of cases. Radiotherapy or chemotherapy have only a slight effect on the survival of patients in whom the tumor has not been resected (median survival 5-6 months) (Safioleas M C et al., Hepatogastroenterology, 2004, 51 (57): 862-868; Jemal A. et al., CA Cancer J Clin, 2002, 52 (1): 23-47; Rosewicz S., et al., Lancet, 1997, 349 (9050): 485-489., Jafari M. et al., Surg Oncol Clin N Am, 2004, 13 (4): 751-760, xi; Kullke M H et al., Curr Treat Options Oncol, 2002, 3 (6): 449-457). Palliative derivations, the placing of biliary or duodenal stents, or even analgesic alcoholization of the celiac plexus are performed in the case of unresectable pancreatic cancers, in particular cancer of the pancreatic head. Patient survival remains very low:
In the case of locally advanced nonmetastatic and unresectable cancer or of metastatic cancer, the current reference treatment is gemcitabine, with which an improvement in clinical signs is observed (pain, transit problems), although the vital prognosis remains poor (7 to 10 months) (Safioleas M C et al., Hepatogastroenterology, 2004, 51 (57): 862-868; Jemal A. et al., CA Cancer J Clin, 2002, 52 (1): 23-47; Rosewicz S., et al., Lancet, 1997, 349 (9050): 485-489., Jafari M. et al., Surg Oncol Clin N Am, 2004, 13 (4): 751-760, xi; Kullke M H et al., Curr Treat Options Oncol, 2002, 3 (6): 449-457).
One of the lines of research for improving the prognosis of pancreatic cancer is to provide an effective treatment applicable in particular to unresectable tumors, with the aim of reducing progression of the disease. Clinical investigation protocols using gene therapy or cell therapy have thus been set up.
   For example, Mulvihill et al. (Gene Therapy, 2001, 8, 308-315) have carried out phase I clinical trials, during which they performed intratumor injection, using a computed tomography (CT) machine, in patients suffering from an unresectable pancreatic carcinoma, of the adenovirus ONYX-015 (dl1520), an adenovirus deleted in the E1B-55 kD gene which preferentially replicates in tumor cells devoid of p53 protein and kills said cells. The injection of adenovirus is well-tolerated but no objective response is demonstrated. The authors emphasize that the intratumor viral replication is not substantial enough and that the adenovirus effectiveness could be optimized by multiplying the ONYX-015 injections or else by combining them with a chemotherapeutic treatment.
   More recently, Hecht et al. (Clinical Cancer Research, 2003, 9, 555-561) modified this protocol, proposing replacement of the CT machine, which is too cumbersome and liable to cause serious complications such as infections or perforations, with ultrasound endoscopy. However, the phase I/II trials carried out by this team revealed no improvement in the effectiveness of the treatment with ONYX-015. In addition, cases of duodenal perforation, due to the endoscope, were observed.
   Other adenoviruses have been tested for treating gastrointestinal tumors. Thus, the team of Sangro et al. (Journal of Clinical Oncology, 2004, 22, 8, 1389-1397) has described the intratumor injection of an adenovirus expressing interleukin 12 (IL-12), called Ad.IL-12, in patients suffering from pancreatic, colorectal or hepatic cancers. These phase I trials have revealed only a moderate antitumor activity. Still directing its research toward the intratumor expression of IL-12 by the Ad.IL-12 adenovirus (also called AFIL-12), the same team has more recently tested the intratumor injection of dendritic cells transfected with the AFIL-12 adenovirus (Mazzolini et al., Journal of Clinical Oncology, 2005, 23, 5, 999-1010). However, additional clinical trials are necessary in order to evaluate the real effectiveness of this treatment.
   Finally, the intratumor administration of an adenoviral vector encoding TNF-α combined with radio-chemotherapy with 5-FU has also been investigated. This trial refers to an antitumor activity at the maximum doses of TNF-α-adenoviral vector (Senzer N. et al., J Clin Oncol, 2004, 22 (4): 592-601). However, the injection of an adenoviral vector is generally accompanied by adverse events such as fever, nausea, lymphopenia, etc.

In addition to strategies making use of adenoviruses, therapies using the “suicide gene”, approach (GDEPT for “gene-derived enzyme prodrug therapy”), i.e. combining the administration of a prodrug and of a gene, the translation product of which metabolizes said prodrug to an active derivative or active derivatives toxic for the tumor cell, have also been developed and tested against gastrointestinal tract cancers such as pancreatic cancer. The article by Günzburg and Salmons (Acta Biochimica Polonica, 2005, 52, 3, 601-607) reviews this approach combined with cell therapy in the context of pancreatic cancer. The authors of this article emphasize the need to develop new cancer treatment strategies that are more effective than the existing treatments. Thus, the authors of this article have proposed administering, in inoperable pancreatic cancers, microcapsules of cellulose sulfate containing recombinant HEK 293 cells expressing cytochrome P450 2B1, in combination with ifosfamide, this being to reduce the effective doses of ifosfamide, which is normally very toxic, owing to a very short half-life of the active plasma formed. The administration of the microcapsules is carried out either directly into the tumor, or into the vascular circulation supplying this tumor, and makes it possible to concentrate the cytochrome P450 at the level of said tumor and therefore to target the action of the ifosfamide metabolites (Löhr et al., The Lancet, 2001, 357, 1591-1592). The clinical trials carried out up until now in humans have shown a doubling of the median survival (Löhr et al., 2001) and a 3-fold increase in survival rate (Löhr et al., 2001; Gunzburg and Salmons, 2005). However, the results obtained are not satisfactory and do not make it possible to significantly increase patient survival (median survival of 10 months and one-year survival rate of 35.7%). More recently, this technique has been extended to other types of cancers, but this remains, for the moment, limited to animal models. Thus, Samel et al. (Cancer Gene Therapy, 2006, 13, 65-73) apply the “targeted chemotherapy” to murine models and show that the injection of said microcapsules combined with administration of ifosfamide in mice developing a human colorectal cancer associated with peritoneal carcinosis can lead to complete remission of the peritoneal tumor.

Cell therapy using microencapsulated cells has the major drawback of using a tumorigenic human cell line (HEK 293 line).

There exists therefore a need to develop new therapeutic anticancer strategies suitable for all cancers and in particular suitable for the treatment of solid tumors, such as gastrointestinal tract tumors.

The inventors have therefore given themselves the aim of providing a new type of anticancer therapy, which, when combined with other therapeutic agents, is effective and less toxic than the treatments currently proposed; this therapy is particularly suitable for gastrointestinal tract cancers, such as inoperable pancreatic cancers.

A subject of the present invention is the use of cells isolated from extramedullary white adipose tissue, selected from the group consisting of the stromal-vascular fraction and a subpopulation of said stromal-vascular fraction consisting of adherent cells, for the preparation of an antitumor medicament.

Adipose tissue exists in various forms in mammals: extramedullary white adipose tissue which represents the main storage organ of the organism, medullary white adipose tissue, the exact role of which is unknown, and thermogenic brown adipose tissue.

Due to its considerable expansion potential which persists throughout the life of the individual, adult white adipose tissue constitutes a source of cells that are abundant and easy to obtain.

This white adipose tissue consists of two cellular fractions:
an adipocyte fraction which represents 30% to 60% of the cells of adipose tissue and is characterized by the accumulation of triglycerides (floating cell fraction). This fraction is very predominantly (99%) composed of differentiated adipocytes and of some contaminating macrophages rich in lipid droplets; and
a nonadipocyte fraction, called stromal-vascular fraction (SVF).

These two cell fractions can be separated by virtue of their difference in density, according to methods such as those described by Björntorp et al. (J. Lipid. Res., 1978, 19, 316-24).

The stromal-vascular fraction, conventionally used to study the differentiation of preadipocytes into mature adipocytes, is a heterogeneous fraction comprising various subpopulations of cells (Planat-Bernard V. et al., Circulation, 2004, 109, 656-663; Zuk P A. et al., Mol. Biol. Cell, 2002, 13, 4279-95; Erickson G R. et al., Biochem. Biophys. Res. Commun., 2002, 290, 763-9; Cousin B. et al., Biochem. Biophys. Res. Commun., 2003, 301, 1016-22; Safford K M. et al., Biochem. Biophys. Res. Commun., 2002, 294, 371-9; international application WO 02/055678 and American application US 2003/0082152). More specifically, the inventors and other teams have previously shown that it is possible to induce the differentiation of the undifferentiated cells of the SVF into various types of differentiated cells. The SVF cells are in fact capable of differentiating into cells expressing specific markers:
hematopoietic cells (international application WO 02/055678, European application EP 1 077 254; U.S. Pat. No. 6,555,374, international application WO 01/62901),
smooth or skeletal muscle cells (international application WO 02/055678, European application EP 1 077 254; U.S. Pat. No. 6,555,374),
cardiac muscle cells (international application WO 02/055678),
endothelial, hepatic, neuronal or astroglial cells (international application WO 01/62901),
pancreatic cells (application US 2003/0124721), or
intraocular stromal cells (international application WO 03/039481).

Furthermore, a subpopulation of homogeneous cells of the SVF, expressing the surface antigens CD13 and HLA ABC, is capable of differentiating into endothelial cells (international application WO 2005/025584).

According to all these publications, the differentiated cells obtained from the SVF cells can be used in tissue repair, reconstituting cell lines and improving the functions of certain tissues. Thus, these differentiated cells can be used, where appropriate:
for reconstituting hematopoietic lines in the context of the treatment of diseases in which a medullary depletion is observed, for example for repopulating the bone marrow of patients who are immunodepressed following an anticancer treatment such as irradiation treatment;
for repairing or reconstructing nerve tissue, for example in the context of the treatment of brain pathologies, such as a stroke, Alzheimer's disease or Parkinson's disease;
for reconstituting hepatic tissue, for example in the context of the treatment of progressive liver degeneration;
for reconstituting cardiac or skeletal muscle tissue, in particular in the context of the treatment of myopathies, cardiomyopathies and pathologies related to muscle degeneration (myocardial infarction);
for improving the functions of pancreatic tissue observed in certain endocrine disorders of the pancreas;
for repairing or reconstructing intraocular tissue in the context of the treatment of corneal tissue or connective tissue lesions, for example after resection of a tumor; and
for completely or partially reconstructing a functional vascular network, in particular in the context of the treatment of ischemia.

For hematopoietic line reconstitution and nerval hepatic tissue repair, international application WO 01/62901 also recommends injecting the undifferentiated stromal cells, and not the differentiated cells.

Surprisingly, the inventors have now demonstrated the antitumor activity of extramedullary white adipose tissue cells selected from the group consisting of the stromal-vascular fraction and a subpopulation of cells isolated from said stromal-vascular fraction, consisting of the cells which, after primary culture of said SVF, adhere to the culture support.

This adherent cell subpopulation will subsequently be referred to, without distinction, as “adherent cell subpopulation”, “adherent cells” or “adherent SVF cells”.

In the subsequent text, the terms “all the cells of the SVF” or “SVF” have the same meaning.

For the purpose of the present invention, the term “SVF” is intended to mean the stromal-vascular fraction comprising all the cells of which it is composed. In the subsequent text, and unless otherwise specified, the expression “cells of the stromal-vascular fraction” includes both the complete stromal-vascular fraction and the adherent cell subpopulation.

The adherent cell subpopulation represents approximately 50% to 60% of the total cell population of the SVF.

Surprisingly, both the SVF and said adherent cell subpopulation significantly slow down tumor progression. This effect is observed not only when the stromal-vascular fraction or said adherent cells are injected locally into the tumor, but also when they are administered systemically, for example parenterally and more specifically intravenously.

Also surprisingly, the SVF can be used for the preparation of an antitumor medicament, with or without expansion, with or without physiological or pharmacological treatment and with or without modification by means of any manipulation of the gene or protein expression profile; the adherent cells can be used for the preparation of an antitumor medicament, with or without physiological or pharmacological treatment and with or without modification by means of any manipulation of the gene or protein expression profile.

Also surprisingly, in addition to the direct antitumor effect of the cells of the stromal-vascular fraction, said cells also exert an antitumor effect indirectly: specifically a medium conditioned by the adherent cell subpopulation inhibits the viability of the cancer cells. This effect is also amplified when said subpopulation of adherent cells is brought into contact beforehand with cancer cells, preferably the cells derived from the cancer to be treated.

The inventors have also shown that, surprisingly, this antitumor effect is associated with an induction in vitro and in vivo, of cell death in the cancer cells.

Remarkably, it is not necessary to induce differentiation of the SVF cells or to cause them to overexpress a particular factor in order for them to exert their antitumor effect. The effect is observed when said SVF cells are used after they have been purified or alternatively after a primary culture and selection of the adherent cells.

According to one advantageous arrangement of said use, said antitumor medicament consists of said isolated cells, before or after culturing.

In accordance with the invention, said subpopulation of adherent cells can be obtained by means of a method comprising:
obtaining the stromal-vascular fraction from extramedullary white adipose tissue;
purifying said stromal-vascular fraction;
isolating the cells of the said subpopulation from said purified fraction by primary culture in a suitable liquid medium, selection of the adherent cells on a culture support (plastic, etc.) by elimination of the nonadherent cells, recovery of the cells after confluence, in a suitable medium, centrifugation and recovery of the pellet.

An example of the method for obtaining these cells is described in the article by Björntorp et al. (mentioned above).

According to an advantageous embodiment of this arrangement, said cells are associated with a pharmaceutically acceptable carrier.

In accordance with the invention, said cells may also be genetically modified:
They may comprise at least one mutation of an autologous gene.
They may contain at least one copy of a heterologous gene.

According to another advantageous embodiment, said cells comprise a heterologous gene, the translation product of which is a protein of therapeutic interest, such as an enzyme capable of metabolizing a prodrug to an active compound or compounds toxic for the tumor cell.

Said genetically modified cells are preferably of human origin.

According to another advantageous arrangement of said use, said antitumor medicament consists of the culture supernatant of said adherent cell subpopulation.

Said supernatant is a primary culture supernatant or a culture supernatant obtained after one or more passages (secondary culture or subsequent cultures).

According to an advantageous mode of this arrangement said supernatant is obtained from a coculture of the adherent cell subpopulation, as defined above, with cancer cells or with cells of a cancer cell line.

The extramedullary adipose tissue is of animal origin or of human origin; preferably, both the cells of the stromal-vascular fraction of the extramedullary adipose tissue and the cancer cells are those of the patient to be treated and have been previously taken from said patient to be treated.

Said supernatant is a primary culture supernatant or a culture supernatant obtained after one or more passages (secondary culture or subsequent cultures).

According to another advantageous embodiment of said use, said cancer is a solid cancer or a liquid cancer.

The term “solid cancer” is intended to mean any cancer affecting the organs such as the liver, the pancreas, the lungs, the kidneys, etc., for which a tumor develops locally and is then dispersed via the blood stream or lymphatic circulation and forms metastases. As opposed to a solid cancer, liquid cancers involve cancers of the blood or of the lymphatic system.

According to a preferred arrangement of this embodiment, said cancer is a solid cancer of the gastrointestinal tract (pancreatic cancer, gastric cancer, colorectal cancer).

The term “cancer of the gastrointestinal tract” is intended to mean tumors and/or cancers of esophagus, of the stomach, of the small intestine, of the large intestine or of the colon and also cancers or tumors of the ancillary glands of the gastrointestinal tract, such as the liver, the gall bladder, the common bowel duct and the pancreas.

Preferably, the antitumor medicament as defined above is particularly suitable for the treatment of pancreatic cancer, and even more preferably for the treatment of inoperable pancreatic cancer.

In such a case, when a coculture of the adherent cell subpopulation and of a cancer cell line is used, the latter is advantageously the Capan-1 pancreatic cell line (ATCC HTB-79).

The antitumor medicament according to the invention is preferably used intratumorally, but it may also be administered by any of the other routes, optionally by multiple routes, in particular intravenously, intra-peritoneally, topically, transdermally, subcutaneously, intraarterially, by the pulmonary route, nasopharyngeally or orally, in solution, in aqueous suspension or as a powder, or in any other pharmaceutically acceptable form.

The effective doses will be determined according to the age, the state of health and the weight of the patient and the type of cancer to be treated.

In accordance with the invention, the use of the antitumor medicament, as defined above, may be combined with other therapies, in particular surgery, radiotherapy, chemotherapy, immunotherapy and differentiating therapies.

The antitumor effect observed, characterized by the slowing down or the inhibition of tumor growth and the induction of cancer cell death (for example, apoptosis), is essentially due to the cells of the cell subpopulation as defined above, i.e. to the cells present in the stromal-vascular fraction and selected on the basis of their adhesion to a solid support (culture support) during primary culture of the SVF cells; however, both the entire stromal-vascular fraction and the cells of said subpopulation can be used in the invention.

The antitumor medicament as defined above (adherent SVF cell subpopulation, or culture supernatant of the cells of said subpopulation) is particularly advantageous for the following reasons:
adipose tissue samples are easy to take, for example by liposuction or by biopsy under local anesthesia;
given its abundance, stocks can be readily formed; in addition, the tissue sampled can be rapidly regenerated in the individual from whom it was taken;
these properties make the cells derived from adipose tissue particularly suitable for a homologous graft (for example, autologous graft) or heterologous graft;
the taking of an adipose tissue sample and the subsequent use thereof for therapeutic purposes should theoretically not encounter any obstacle of ethical type since the taking of the sample is relatively noninvasive and many adipose tissue samples are currently destroyed. In addition, it should be noted that the hospitalization time necessary for taking said sample is short (in particular, there is no need for recourse to cytapheresis or to general anesthesia);
it is possible to maintain and multiply, or even immortalize, the cells in vitro in a defined medium; moreover, these cells can be transfected and can be used for expressing a heterologous gene, all the more so since they have a strong secreting capacity. It is therefore possible to use them for expressing a therapeutic protein, for example an enzyme capable of converting a prodrug to an active drug.

A subject of the present invention is also the use of an antitumor agent as defined above, namely selected from the group consisting of extramedullary white adipose tissue cells as defined above, the culture supernatant of said cells or the supernatant of a coculture of said cells with cancer cells or cells of a suitable cancer cell line, as an adjuvant in anticancer therapy.

Preferably, said supernatant is a primary culture supernatant or a primary coculture supernatant. It may also be a culture or coculture supernatant obtained after one or more passages.

A subject of the present invention is also the use of an antitumor agent as defined above, namely selected from the group consisting of extramedullary white adipose tissue cells as defined above, the culture supernatant of said cells or the supernatant of a coculture of said cells with cancer cells or cells of a suitable cancer cell line, for screening in vitro, for other antitumor medicaments, in particular capable of acting in synergy with said cells or supernatant of said cells.

Preferably, said supernatant is a primary culture supernatant or a primary coculture supernatant. It may also be a culture or coculture supernatant obtained after one or more passages.

In addition to the above arrangements, the invention also comprises other arrangements which will become clear from the description which follows, which refers to exemplary embodiments of the method which is the subject of the present invention, and also to the attached drawings, in which:

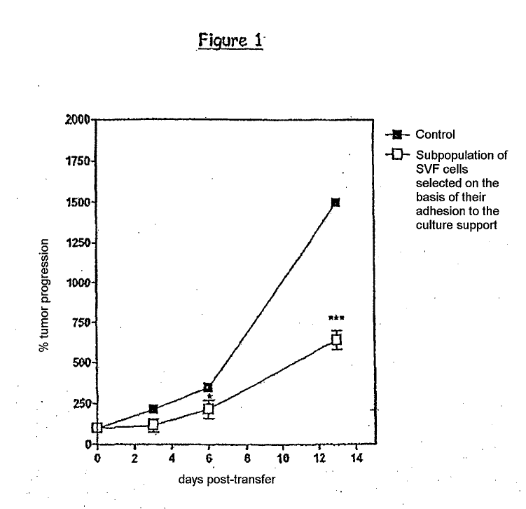

FIG. 1: in vivo determination of tumor progression after intratumoral injection of the SVF cell subpopulation selected on the basis of their adhesion to the culture support (□) or of PBS (▪). x-axis: number of days after injection; y-axis: percentage tumor progression. *: p<0.05, ***: p<0.001.

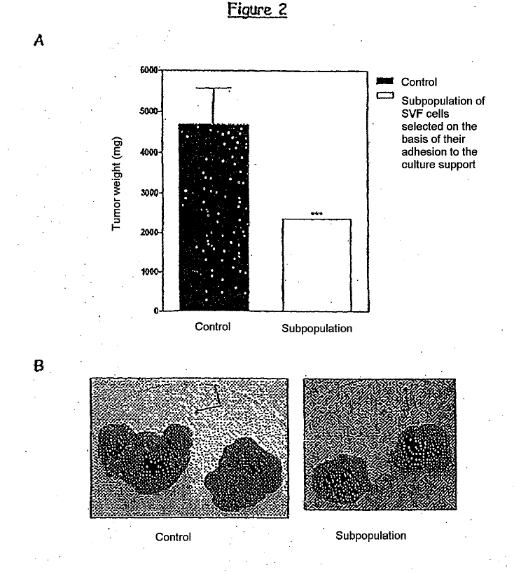

FIG. 2: Decrease in pancreatic tumor weight in vivo after intratumoral injection of the adherent cell subpopulation. A: determination of the tumor weight (mg) after intratumoral injection of cells of the stromal-vascular fraction (white) or of PBS (control) (black); ***: p<0.001. B: photograph of tumors taken after intratumoral injection of the adherent cell subpopulation (right) or of PBS (left).

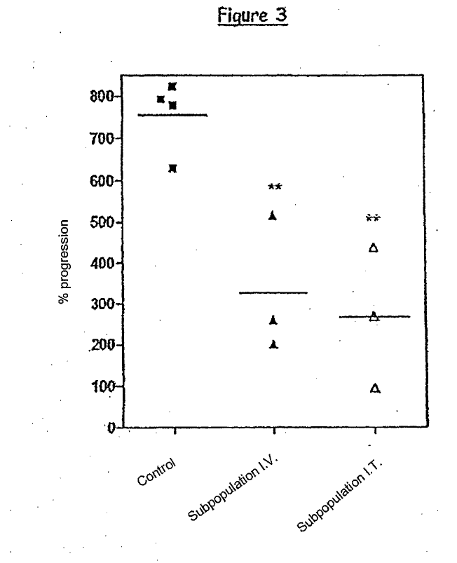

FIG. 3: in vivo determination of the percentage tumor progression after intratumoral injection (I.T.; Δ) or systemic injection (I.V.; ▴) of the adherent cell subpopulation, or either intratumoral or systemic injection of PBS (control; ▪). **: p<0.01.

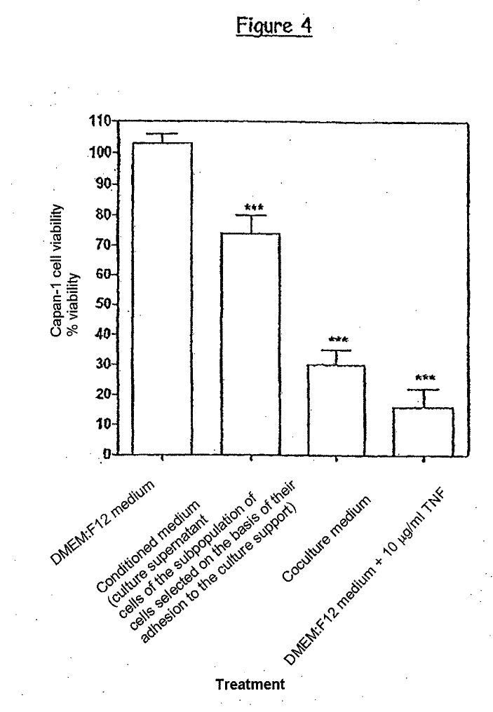

FIG. 4: in vitro determination of the percentage viability of Capan-1 cells treated with DMEM:F12 OK medium, with culture supernatant of said subpopulation of cells (conditioned medium), with coculture supernatant from said subpopulation of cells and Capan-1 cells (coculture medium) or else with DMEM:F12 medium supplemented with 10 μg/ml of TNF-α. *** p<0.001.

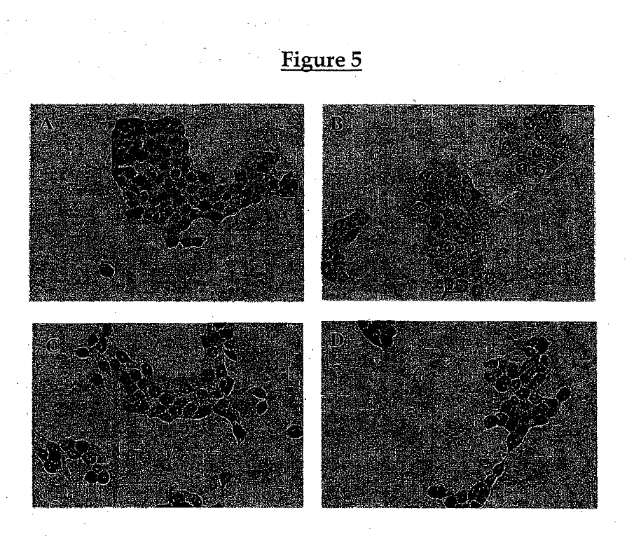

FIG. 5: in vitro determination of Capan-1 cell apoptosis induced by the culture supernatant of cells of the adherent cell subpopulation and by the coculture supernatant of the cells of the adherent cell subpopulation with the Capan-1 cells. A: Capan-1 cells placed in the presence of DMEM:F12 OK medium (negative control). B: Capan-1 cells treated with the culture supernatant of cells of the adherent cell subpopulation. C: cells treated with the coculture supernatant of cells of the adherent cell subpopulation with the Capan-1 cells, according to a Capan-1/adherent cell ratio of 5/1; D: cells treated with the coculture supernatant of cells of the adherent cell subpopulation with the Capan-1 cells, according to a Capan-1/adherent cell ratio of 1/1.

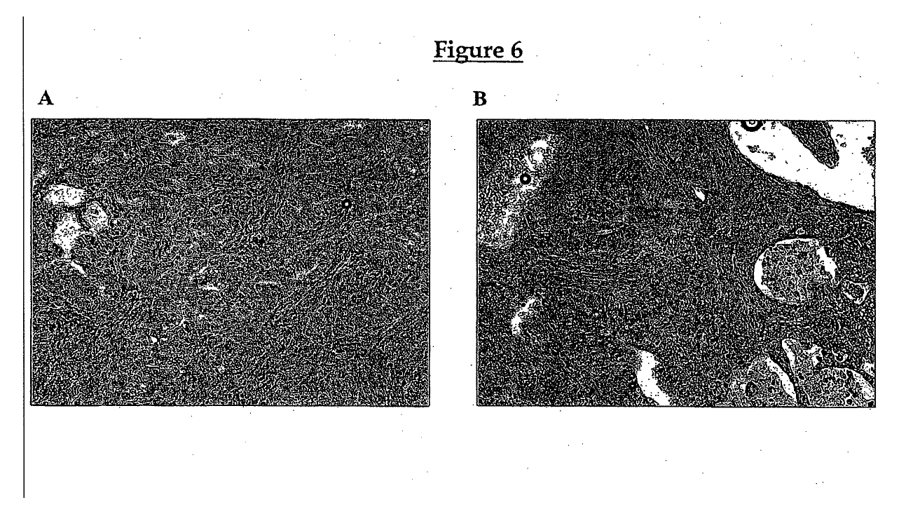

FIG. 6: in vivo determination of the cancer cell apoptosis after intratumoral injection of cells of the adherent cell subpopulation. Section of control pancreatic tumor (A) or a pancreatic tumor after injection of the cells of the adherent cell subpopulation (B).

It should, however, be clearly understood that these examples are given only by way of illustration of the subject matter of the invention, of which they in no way constitute a limitation.

### EXAMPLE 1

### Materials and Methods

### 1) Media Used

The DMEM F12-OK medium comprises, per 500 ml of DMEM F12 (Gibco reference 31330 038), 5 ml of ASP (ready-to-use solution of antibiotics+antifungal: 0.25 μg/ml amphotericin, 100 μg/ml streptomycin, 100 μg/ml penicillin G (Sigma reference A7292), 0.5 ml of 16 mM biotin (0.016 mM final concentration) (Sigma reference B4639), 0.5 ml of 18 mM pantothenic acid (Sigma P5155) (final concentration 0.018 mM), 0.5 ml of 100 mM ascorbic acid (Sigma A4034) (final concentration 100 μM).
The digestion buffer contains DMEM F12-OK, 2% BSA (bovine serum albumin) and 2 mg/ml of collagenase (Sigma reference) in a proportion of 10 ml of digestion medium per 3 g of tissue. This buffer is filtered through 0.2 μm filters (Acrodisc PF 0.8/0.2 μm, ref PALL6224187, VWR).
The lysis buffer comprises 100 ml of solution A (2.08 g of Tris buffer, pH 7.65, in 100 ml of sterile H₂O) and 900 ml of solution B (8.3 g of NH₄Cl in 1000 ml of sterile H₂O).
The complete RPMI medium is prepared from RPMI (Roswell Park Memorial Institute) 1640 medium (Gibco/Invitrogen (ref 21875034)) supplemented with 10% of newborn calf serum (NCS Gibco 18010-159), 100 μ/ml of penicillin, 100 μ/ml of streptomycin and 0.25 μg/ml of fungizone (Invitrogen, 15240-096).
The PBS solution comes from Gibco (ref 14200-067).
The trypsin/EDTA solution comes from Gibco (ref 25300-054).

### 2) Obtaining the Stromal-Vascular Fraction

### Digestion

The adipose tissue comes from patients who undergo a dermolipectomy or liposuction:

### For a Dermolipectomy:

The required amount of adipose tissue is weighed into a sterile dish, and then all the work is carried out under a culture hood. The tissue is subjected to mechanical dissociation through being chopped up very finely with scissors, and the fragments obtained are rinsed with PBS.

The adipose tissue sample taken is dissected under a microscope in sterile dishes containing PBS, so as to remove all traces of muscle tissue, and then digested at 37° C. for 30 min, in digestion medium. The digestion is accelerated by manual agitation every 10 min.

### For Liposuction:

The protocol is identical, with the exception of the step of mechanical dissociation of the tissue with a pair of scissors, which is not necessary.

### Purification of the Stromal-Vascular Fraction (SVF)

After removal of the undigested fragments by filtration (25 μm filters) (PA 25/21, 25 μm, Tissage de Tissue Techniques, Sailly-Saillisel), the mature adipocytes are separated from the pellet containing the SVF cells by centrifugation (600 g, 10 min). The stromal-vascular cells thus isolated (pellet) are resuspended in 2 ml of DMEM:F12 medium+10% NCS (newborn calf serum) and counted (manual counting on a grid cell counter or a Coulter particle counter) and the cells are resuspended in the same medium. The same volume of lysis buffer is added and the cell suspension is centrifuged for 5 minutes at 1600 rpm (500 g). The supernatant is removed and the pellet is taken up in DMEM:F12 OK (from 500 μl to 1 ml depending on the size of the pellet).

### 3) Cell Culture and Obtaining the Cell Subpopulation

After obtaining the crude stromal-vascular fraction (SVF), according to the method described above, the cells are seeded into 25 cm² flasks (Nunc, angled neck and filtering cap, ref 055422, Dominique Dutscher) at a rate of 5×10⁵ to 10⁶ cells per dish and cultured in the DMEM:F12 OK medium. The cells are rinsed with PBS the day after having been placed in culture in order to remove all the dead and/or nonadherent cells, and are then cultured for 4 to 7 days in DMEM:F12 OK medium+10% NCS.

After confluence (i.e. after 4 to 7 days of culture), the cells are rinsed with PBS so as to remove all traces of medium. The cells are detached using a solution of trypsin/EDTA and then counted using a cell counter (Coulter ZI). The cell suspension is centrifuged for 5 min at 1600 rpm (500 g), and the pellet is then taken up in a suitable volume of PBS, so as to have a concentration of the order of 10⁶ cells/100 μl.

4) Capan-1 Line and Murine Model of Human Pancreatic cancer

A murine model of human pancreatic cancer is set up as indicated below. This model has an ectopic tumor at the subcutaneous level.

Capan-1 Line

The Capan-1 cells are derived from liver metastases of human pancreatic adenocarcinomas (ATCC HTB-79). The Capan-1 cells are routinely cultured in complete RPMI culture medium at 37° C. and 5% CO₂, in culture flasks (reference BD Falcon T-175 353028), and are subcultured when they reach 70 to 80% confluence.

Swiss nu/nu Mice

The athymic female Swiss Nude (nu/nu) mice (Charles River) are 6 to 8 weeks old at the time of the experiment. After reception, the Swiss nu/nu mice are tattooed on the ear for subsequent identification, and then acclimatized to the rearing conditions for 1 week in an A2 environment before experimentation (zootechnics department of IFR31, Toulouse), according to diurnal and nocturnal cycles of 12 hours. The Swiss nu/nu mice are housed at 5 per cage on a ventilated cage rack system.

Injection of Capan-1 Cells

### Preparation of Injectable Cell Suspensions

The Capan-1 cell culture medium is drawn off and the cells are rinsed with 10 ml of sterile PBS. After incubation for 5 minutes at ambient temperature, the PBS is drawn off and the cells are dissociated with 3 ml of a solution of trypsin/EDTA, for 5 minutes at 37° C. The cells are then taken up in 7 ml of complete medium and then dissociated with a pipette after 10 cycles of drawing up/blowing back, and then harvested in a sterile 50 ml tube (Falcon Blue Max 50 ml 352070). The cells are counted using a Coulter Z.I. cell counter. The equivalent of 10⁷ Capan-1 cells per mouse to be injected is centrifuged for 5 min at 1400 rpm (200 g) under sterile conditions.

The supernatant is removed, and the pellet is then taken up in 25 ml of complete medium and then dissociated with a pipette after 10 cycles of drawing up/blowing back in order to remove all traces of trypsin. After centrifugation for 5 min at 1400 rpm (200 g), the supernatant is removed and the pellet is subjected to 3 cycles of washing in RPMI medium in order to remove all traces of serum. Finally, 10⁷ Capan-1 cells are taken up in 100 μl of sterile PBS for cell culture.

### Implantation of Capan-1 Tumors

The mice, strictly handled under an MSC (microbiological safety cabinet) hood, are identified by virtue of their tattoo, weighed, and then anesthetized with 0.1% isofluorane (Aerrane, Baxter) for 5 minutes before handling. This general anesthesia protocol enables the individuals to be handled comfortably and ensures reproducibility of the results, while at the same time preventing experimental artefacts subsequent to the stress of the animal. After having observed that the animal has gone to sleep, the Capan-1 cells (100 μl ) are injected subcutaneously into the left flank of the animal, using a 0.3 ml 29G by 33 mm tuberculin syringe with no dead space, at a speed of 1 ml/h. The injection site is disinfected, and the mice are reconditioned in cages with clean litter. Under these experimental conditions, the mice come round 5 to 7 minutes after anesthesia. The vital signs of the injected mice are analyzed macroscopically 24 and 48 h following the operation. Under these conditions, the mortality rate measured is 0%.

### 5) Injection of the Cells of the Adherent Cell Subpopulation

The injection of the adherent cell subpopulation is carried out 11 to 14 days after implantation of the Capan-1 tumors into the athymic Swiss nu/nu mice. The average tumor volume is then 250±18 mm³. As indicated above, the mice, strictly handled under an MSC hood, are identified by virtue of their tattoo, weighed, and then anesthetized with 0.1% isofluorane for 5 minutes before handling. After having observed that the animal has gone to sleep, the cells (5×10⁵ in 50 μl of PBS) are injected either directly into the tumor, or into the caudal vein.

For the intratumor graft, the cells are injected using a 0.3 ml 29G by 33 mm tuberculin syringe with no dead space, at a speed of 1 ml/h. In this context, the cells are in an environment of only pancreatic tumor cells; they are not in contact with the healthy pancreatic tissue. For the intravenous injection, the mice are placed in an injection chamber heated to 50° C. in order to facilitate dilation of the caudal vein, and the cells are injected using a lymphography device with no dead space, of caliber 29G, made of PVC.

In the two cases, 50 μl of sterile PBS are injected into the control mice, either intratumorally or systemically. The injection site is disinfected, and the mice are reconditioned in cages with clean litter.

### 6) Statistical Analysis

The statistical analysis is carried out using the Graphpad Instat V3.05 software. The analysis of variance is carried out using the ANOVA unilateral test completed by a Student-Newman-Keuls multiple comparison test. A probability below 0.05 is considered to be statistically significant.

### 7) Detection of Apoptosis by Means of the TUNEL Technique

Apoptosis of the cancer cells is detected on Capan-1 cell cultures or on sections of pancreatic tumors originating from the murine model described in Example 1.4, by means of the TUNEL technique (terminal deoxynucleotidyl transferase mediated dUTP-biotin nick end labeling). This technique is based on the incorporation of labeled nucleotides at the free 3′OH ends of the DNA fragments generated during apoptosis (Gavrieli et al., 1992, the Journal of Cell Biology, Vol. 119, No. 3, pages 493-501). It is carried out here using the ApopDETEK kit (EnzoDiagnostic, NY, USA) according to the manufacturer's recommendations.

### EXAMPLE 2

### Antitumor Effect, In Vivo, of the Cells Derived from Adipose Tissue

The antitumor effect of the cells of the stromal-vascular fraction of extramedullary adipose tissue is evaluated when said cells are injected into the tumor or systemically in Swiss nu/nu mice developing a pancreatic tumor derived from Capan-1 cells.

### 1) Intratumoral Injection

The conditions for preparing the mice, for injecting the Capan-1 cells and for injecting the adherent cell subpopulation are described in Example 1.

After injection of the adherent cell subpopulation, the weight of the mice and the growth of the tumors are measured and recorded every 2 days, up until 14 days after the injection of said cells, the tumor growth being measured in situ on the live animal. The lengths (L) and widths (l) of the Capan-1 tumors are measured using a calliper rule, according to the following calculation: Tumor volume (mm³)=L² (mm)×l (mm)×0.52

The percentage tumor progression is evaluated 3, 6 and 13 days after intratumoral injection of said cells or of PBS (control) according to the following formula:

% tumor progression at time t=[(tumor volume at time t)/(tumor volume at t₀)]×100; t₀ corresponding to the injection of the adherent cell subpopulation or PBS.

The experiment is stopped at 10 to 15 days after transfer of the adherent cell subpopulation (21 to 39 days after tumor implantation) due to the exponential progression of the control tumors, to the ulceration of said tumors and to the high risk of death of the mice carrying these tumors.

In addition to the determination of the tumor progression by means of measuring the tumor size, the tumor weight is also determined. For this, the mice are sacrificed 13 days after intratumoral injection of the cells and the tumors are removed, weighed and photographed.

The values obtained, on the one hand, for the tumor progression and, on the other hand, for the tumor weight measurement are representative of three independent experiments, corresponding to three different preparations of the adherent cell subpopulation, and 5 mice per group.

The results of the tumor progression inhibition test are given in FIG. 1. They show a drastic and significant decrease in the size of the Capan-1 tumors of 46%±13%, 38%±8% and 57%±14% respectively 3, 6 and 13 days after injection of the subpopulation cells. These data show an antitumor effect which manifests itself through a drastic decrease in the progression of the Capan-1 pancreatic tumors after intratumoral transfer of cells. The experiment could not be continued due to the exponential growth and the ulceration of the control tumors.

The results relating to the measurement of tumor weight and size 13 days after injection of the adherent cell subpopulation are given in FIG. 2. They show a decrease of 50%±0.1% in the weight of the Capan-1 tumors after intratumoral injection of said cells. These results are in agreement with those relating to the percentage tumor progression, obtained from the extrinsic measurement of the tumor size (FIG. 1) and therefore confirm the antitumor role of the various cells when the latter are injected intratumorally.

### 2) Systemic Injection

In parallel with these studies of decrease in tumor progression by intratumor injection of the adherent cell subpopulation, the inventors administered these cells via the blood, in order to determine whether they could migrate to the site of the tumor and exert their effect at said site.

The conditions for preparing the mice and for injecting the Capan-1 cells, the adherent cell subpopulation and the PBS are described in Example 1. More specifically, the inventors compared the effect of the subpopulation cells administered either directly into the Capan-1 tumors (intratumoral injection) or in the caudal vein (systemic injection). The tumor size is measured as indicated above, 3 days after transfer of the subpopulation cells. The values obtained are representative of 2 independent experiments corresponding to two different cell preparations, and 3 or 4 mice per group. The results are given in FIG. 3. They show that the size of the inhibition of tumor progression measured 3 days after transfer of the SVF cells, when the latter are injected into the caudal vein of the mice carrying Capan-1 tumors, is comparable to that observed when the cells are directly administered into the tumor (−56%±22% vs −65%±26%).

These results were in favor of an antitumor effect in vivo, of the cells of the subpopulation when said cells are injected systemically.

All the results given in Example 2, showing inhibition of tumor progression by the cells of the subpopulation injected locally into the tumor or else administered systemically in the caudal vein, strongly suggest an antitumor role for the cells of the stromal-vascular fraction of extramedullary white adipose tissue.

In addition, these results show targeting of the pancreatic tumor by the SVF cells selected on the basis of their adhesion to the culture support, when said cells are administered remotely in the systemic blood stream.

### EXAMPLE 3

### Measurement of Cell Viability

The inventors also measured the viability of the Capan-1 cells in the presence of culture supernatant of the adherent cell subpopulation in vitro, in order to confirm and validate the results obtained in vivo (Example 2).

The Capan-1 cells are seeded in sextuplicate into 96-well flat-bottomed culture dishes (Nunc 167008), at a rate of 25 000 cells per well, in a final volume of 100 μl, and 48 h later, the cells are rinsed and then treated:
with 100 μl of serum-free RPMI medium (negative control, not represented in FIG. 4);
with 100 μl of complete RPMI medium (positive control, not represented in FIG. 4);
with 100 μl of DMEM:F12 medium supplemented with 10 μg/ml of TNF-α (positive control for inhibition of viability);
with 100 μl of DMEM:F12 OK medium;
with 100 μl of culture supernatant of cells of the adherent cell subpopulation; or
with 100 μl of coculture supernatant of cells of the adherent cell subpopulation and Capan-1 cells.

The culture supernatant of cells of the adherent cell subpopulation is obtained after 48 hours of culture in the DMEM:F12 OK medium; the coculture supernatant of cells of the adherent cell subpopulation and Capan-1 cells is obtained after 48 hours of a culture of adherent cells and Capan-1 cells in the DMEM:F12 OK medium, according to an initial Capan-1 cell/adherent cell ratio of 5/1 or 1/1 (respectively, coculture supernatant 1 and coculture supernatant 2).

Two days (48 h) later, the cell viability is measured using the cell titer 960 AQueous One Solution Cell Proliferation Assay kit (Promega G3582). The values obtained are representative of 3 independent experiments corresponding to three different subpopulation cell preparations.

The results are given in FIG. 4. They show that the medium conditioned by the adherent cell subpopulation (culture supernatant) is capable of significantly inhibiting the viability of the Capan-1 cells. Furthermore, the coculture supernatant of the adherent cell subpopulation with Capan-1 cells has a more pronounced inhibitory effect on the Capan-1 cell viability than the supernatant conditioned by the adherent cell subpopulation alone, which shows that these cells are capable of reacting to the presence of pancreatic cancer cells.

These results indicate that a medium conditioned by the cells of the stromal-vascular fraction of extramedullary white adipose tissue has an antitumor effect, it being possible for said effect to be amplified when the cells are activated by cancer cells such as the cells of the Capan-1 line.

### EXAMPLE 4

### Induction of Apoptosis

The inventors investigated whether the antitumor effect of the cells of the stromal-vascular fraction of extramedullary white adipose tissue and of the culture and coculture supernatants is due to the induction of apoptosis of the cancer cells.

### 1) In Vitro Induction of Capan-1 Cell Apoptosis

The Capan-1 cells are seeded in triplicate and cultured on 4-well Labtecks (Dutscher), at a rate of 50 000 cells per well, in a final volume of 500 μl, then rinsed, and treated with:
500 μl of DMEM:F12 OK medium or of 10% RPMI medium;
500 μl of culture supernatant of cells of the adherent cell subpopulation;
500 of coculture supernatant 1;
500 μl of coculture supernatant 2.

The culture supernatant of cells of the adherent cell subpopulation is obtained as indicated in Example 3.

24 hours later, the apoptosis is evaluated by means of the TUNEL technique as indicated in Example 1.7.

The results are given in FIG. 5.

Nuclear labeling of the Capan-1 cells treated with the culture supernatant of the cells of the adherent cell subpopulation is observed (FIG. 5B). This labeling is not observed for the Capan-1 cells placed in the presence of DMEM:F12 OK medium or of 10% RPMI (FIG. 5A). For the Capan-1 cells treated with coculture supernatant 1, the nuclear labeling is more intense (FIG. 5C) and the intensity is further increased by treatment with coculture supernatant 2, for which condensation of the cytoplasm is also observed (FIG. 5D). These results show a quantitative increase in DNA fragmentation in the presence of coculture supernatant, and therefore an increase in Capan-1 cell apoptosis.

These results therefore show that the culture supernatant of the cells of the adherent cell subpopulation and the coculture supernatant of the cells of the adherent cell subpopulation are capable of inducing Capan-1 cell apoptosis in vitro.

### 2) In Vivo Induction of Apoptosis of Pancreatic Cancer Cells

The conditions for preparing the mice, for injecting the Capan-1 cells and for intratumoral injection of the adherent cell subpopulation are described in Example 1.

Five days after injection of the cells of the adherent cell subpopulation, pancreatic tumor biopsies are taken and then sections are prepared. For the negative control, tumor sections are prepared from mice having not received cells of the adherent cell subpopulation. Apoptosis is detected in these two types of preparation by means of the TUNEL technique as indicated in Example 1.7.

The results are indicated in FIG. 6.

No labeling is detected in the sections of tumors originating from the control mice (FIG. 6A). On the other hand, nuclear labeling is observed for many cancer cells originating from mice having received the injection of cells of the adherent cell subpopulation (FIG. 6B), thereby showing that these cells have entered into apoptosis.

These results indicate that the cells of the adherent cell subpopulation are capable of inducing cancer cell apoptosis in vivo.

1. A method of preparing an antitumor medicament comprising isolating cells from extramedullary white adipose tissue, selected from the group consisting of the stromal-vascular fraction and a subpopulation of said stromal-vascular fraction consisting of adherent cells, and providing said cells in a medicament.

2. The method as claimed in claim 1, wherein said adherent cell subpopulation are obtained by means of a method comprising:
obtaining the stromal-vascular fraction from extramedullary white adipose tissue;
purifying said stromal-vascular fraction;
isolating said subpopulation of cells from said purified fraction by primary culture in a suitable liquid medium, selection of the adherent cells on a culture support, recovery of the cells after confluence, in a suitable medium, centrifugation and recovery of the pellet.

3. The method as claimed in claim 1, wherein said cells are associated with a pharmaceutically acceptable carrier.

4. The method as claimed in claim 1, wherein said cells comprise a heterologous gene, the translation product of which is a protein of therapeutic interest, to an active compound or compounds toxic for the tumor cell.

5. The method as claimed in claim 1, wherein said antitumor medicament consists of the culture supernatant of said adherent cell subpopulation.

6. The method as claimed in claim 5, wherein said supernatant is obtained from a coculture of said cell subpopulation with cancer cells or with cells of a cancer cell line.

7. The method as claimed in claim 1, wherein said cancer is a solid cancer or a liquid cancer.

8. The method as claimed in claim 7, wherein said cancer is a solid cancer of the gastrointestinal tract.

9. The method as claimed in claim 1, wherein said step of isolating comprises isolating the culture supernatant of said cells or the supernatant of a coculture of said cells with cancer cells or cells of a suitable cancer cell line.

10. (canceled)

11. A method of treatment comprising administering to a subject a composition comprising cells isolated from extramedullary white adipose tissue, selected from the group consisting of the stromal-vascular fraction and a subpopulation of said stromal-vascular fraction consisting of adherent cells.

12. The method as claimed in claim 11, wherein said step of administering comprises injecting the composition locally into a tumor.

13. The method as claimed in claim 11, wherein said step of administering comprises administering the composition to the subject systemically.

14. The method as claimed in claim 11, carried out in combination with other therapies selected from the group consisting of surgery, radiotherapy, chemotherapy, immunotherapy and differentiating therapy.

15. A method of in vitro screening of an antitumor medicament comprising:
contacting an antitumor agent selected from the group consisting of isolated cells from extramedullary white adipose tissue selected from the group consisting of the stromal-vascular fraction and a subpopulation of said stromal-vascular fraction consisting of adherent cells, and the culture supernatant of said cells with cancer cells or cells of a suitable cancer cell line,
adding said antitumor medicament to be screened, and
measuring the synergy between said isolated cells or supernatant of said cells and said antitumor medicament on said cancer cells or cells of a suitable cancer cell line.
